(19) United States
(12) Patent Application Publication
(10) Pub. No.: US 2015/0344975 A1
(43) Pub. Date: Dec. 3, 2015

(54) COMPOSITIONS AND METHODS FOR DETECTING, EXTRACTING, VISUALIZING, AND IDENTIFYING PROTOMYXZOA RHUEMATIC

(71) Applicants: Stephen E. Fry, Scottsdale, AZ (US); Jeremy Ellis, Mesa, AZ (US)

(72) Inventors: Stephen E. Fry, Scottsdale, AZ (US); Jeremy Ellis, Mesa, AZ (US)

(21) Appl. No.: 13/566,972

(22) Filed: Aug. 3, 2012

Related U.S. Application Data
(60) Provisional application No. 61/514,845, filed on Aug. 3, 2011.

(51) Int. Cl. C12Q 1/68

(52) U.S. Cl. CPC C12Q 1/6893

(57) ABSTRACT

Disclosed are compositions, kits, and methods for detecting, extracting, visualizing, and identifying Protomyxzoa Rhuematic.

### CROSS REFERENCE TO RELATED APPLICATION

This application claims the benefit of the earlier U.S. Provisional Patent Application, Ser. No. 61/514,845, filed Aug. 3, 2011, now pending, the entire disclosure of which being hereby incorporated entirely herein by reference.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 3,142,063 byte ASCII (text) file named “Sequence Listing” created on Aug. 2, 2011.

### BACKGROUND

1. Technical Field

This document relates to compositions and methods for detecting, extracting, visualizing, and identifying Protomyxzoa Rhuematic.

2. Background

Rheumatic and inflammatory diseases have had a long history of links with infectious agents ranging from molecular mimicry effects to the direct activity of human pathogens.

### SUMMARY

Aspects of this document may comprise, and implementations may include, one or more or all of the components and steps set forth in the appended CLAIMS, which are hereby incorporated by reference.

One aspect of this document relates at least to an isolated DNA coding for a polypeptide, the isolated DNA having any one of the nucleotide sequences set forth in SEQ ID NO:1 through SEQ ID NO:8454 which are identifying for Protomyxzoa Rhuematic.

Another aspect of this document relates at least to an isolated oligonucleotide (by way of non-limiting example, a forward primer, a reverse primer, or a probe such as a molecular beacon) capable of detecting a unique biomarker (a fragment of DNA sequence that causes disease or is associated with susceptibility to disease) for Protomyxzoa Rhuematic, such as any one of the nucleotide sequences set forth in SEQ ID NO:1 through SEQ ID NO:8454.

Specifically, aspects of this document relate at least to identifying any one of the nucleotide sequences set forth in SEQ ID NO:1 through SEQ ID NO:8454 for Protomyxzoa Rhuematic using hot-start polymerase chain reaction (PCR) and employing specific and/or universal primers.

Still another aspect of this document relates to methods useful for detecting Protomyxzoa Rhuematic from a sample. Such methods may comprise aligning nucleotide sequences pair wise and determining the percent identities (percentage of identical matches) between the universal primers and the sample to be tested. In particular implementations, a reaction mixture or a kit may be provided comprising a first isolated oligonucleotide (a forward primer, in particular implementations), and a second isolated oligonucleotide (a reverse primer, in particular implementations).

Yet another aspect of this document relates to methods useful for detecting Protomyxzoa Rhuematic from a sample. Such methods may comprise determining whether a sample (by way of non-limiting examples, a blood sample, or other a biological sample, such as a swab specimen) contains Protomyxzoa Rhuematic, or has an increased likelihood of containing Protomyxzoa Rhuematic. Such a method may comprise the following: (a) providing a vessel containing a composition, wherein the composition contains first and second primers and a nucleic acid from the sample, wherein the composition is capable of amplifying by a polymerase chain reaction a segment of the nucleic acid to produce an amplicon, wherein production of the amplicon is primed by the first and second primers, wherein the first primer is capable of hybridizing under highly stringent hybridization conditions to a polynucleotide consisting of the nucleotide sequence of one of: any one of the nucleotide sequences set forth in SEQ ID NO:1 through SEQ ID NO:8454; ATGGCTCATTATATCAGTTATAGT; and CCATGCATGTCTAAGTATA; and wherein the second primer is capable of hybridizing under highly stringent hybridization conditions to a polynucleotide consisting of the nucleotide sequence of one of: any one of the nucleotide sequences set forth in SEQ ID NO:1 through SEQ ID NO:8454; and GTTATTATGATTCACCAAACAAG; (b) incubating the vessel under conditions allowing production of the amplicon if the sample contains Protomyxzoa Rhuematic; and (c) determining that the sample contains pathogen Protomyxzoa Rhuematic if the amplicon is detected or that the sample has an increased likelihood of containing pathogen Protomyxzoa Rhuematic if the amplicon is detected, or determining that the sample does not contain Protomyxzoa Rhuematic if the amplicon is not detected or that the sample does not have an increased likelihood of containing Protomyxzoa Rhuematic if the amplicon is not detected.

The vessel may also contain an oligonucleotide probe capable of detecting the amplicon if the amplicon is produced, the probe consisting of the nucleotide sequence of FAM-ACATCCTTT/ZEN/CCGTGAGGTCAGGAGTT-3IABkFQ.

The sample may be an extracted sample obtained by one of an expanded extraction method and an enrichment, expanded extraction method as described below.

The foregoing and other aspects, features, and advantages will be apparent to those of ordinary skill in the art from the DESCRIPTION and DRAWINGS, and from the CLAIMS.

### BRIEF DESCRIPTION OF DRAWINGS

The patent or application file contains at least one drawing executed in color. Copies of this patent or patent application publication with color drawing(s) will be provided by the Office upon request and payment of the necessary fee.

Implementations will hereinafter be described in conjunction with the appended DRAWINGS, where any like designations denote like elements.

FIG. 1 is a dual Dual Hoechst and EtBr DNA stain (1000×) revealing large amorphous clusters, 20-100 μm in diameter, including bacteria (yellow arrows) and organisms, Protomyxzoa Rhuematic, (white arrows) with varying dye permeability, and red blood cells (arrow heads).

FIGS. 2 and 3 are PAS and Hoechst stains respectively revealing that the extracellular matrix material associated with the clusters of FIG. 1 contain both DNA and polysaccharides.

FIG. 4 show the gel results for a detection study revealing a mixed population of organisms, including Proteobacteria (primarily Ralstonia spp.), fungi, and sequences suggestive of a novel protozoan species Protomyxzoa Rhuematic.

FIG. 5 depicts two panels that are modified May-Grünwald stains (1000×), the Left Panel showing ring forms within red blood cells (yellow arrow) found in association with biofilms and may represent part of the organisms life cycle, and the Right Panel showing biofilm detection with embedded organisms (yellow arrow) which are rarely detected by traditional means.

FIG. 6 depicts two panels that are fluorescent DNA stains revealing the presence of biofilms in patients with chronic inflammatory and neurologic disease, the Left Panel showing a high magnification (400×) image revealing irregular size organisms bound by a DNA rich biofilm matrix, and the Right Panel showing a low magnification (100×) image demonstrating the lymphocytic response with adherent white blood cells (yellow arrows) to a large biofilm cluster.

### DESCRIPTION

### Overview, Terminology, Case Study, and Evidence

The majority of genes act by specifying polypeptide chains that form proteins. Proteins in turn make up living matter and catalyze all cellular processes.

Chemically, a genome is composed of deoxy-ribonucleic acid (“DNA”). Each DNA molecule is made up of repeating units of four nucleotide bases—adenine (“A”), thymine (“T”), cytosine (“C”), and guanine (“G”)—which are covalently linked, or bonded, 2 together via a sugar-phosphate, or phosphodiester, backbone. DNA generally exists as two DNA strands intertwined as a double helix in which each base on a strand pairs, or hybridizes, with a complementary base on the other strand: A pairs with T, and C with G.

The linear order of nucleotide bases in a DNA molecule is referred to as its “sequence.” The sequence of a gene is thus denoted by a linear sequence of As, Ts, Gs, and Cs. “DNA sequencing” or “gene sequencing” refers to the process by which the precise linear order of nucleotides in a DNA segment or gene is determined. A gene's nucleotide sequence in turn encodes for a linear sequence of amino acids that comprise the protein encoded by the gene. Most genes have both “exon” and “intron” sequences. Exons are DNA segments that are necessary for the creation of a protein, i.e., that code for a protein. Introns are segments of DNA interspersed between the exons that, unlike exons, do not code for a protein.

The creation of a protein from a gene comprises two steps: transcription and translation. First, the gene sequence is “transcribed” into a different nucleic acid called ribonucleic acid (“RNA”). RNA has a chemically different sugar-phosphate backbone than DNA, and it utilizes the nucleotide base uracil (“U”) in place of thymine (“T”). For transcription, the DNA double helix is unwound and each nucleotide on the non-coding, or tem-plate, DNA strand is used to make a complementary RNA molecule of the coding DNA strand, i.e., adenine on the template DNA strand results in uracil in the RNA molecule, thymine results in adenine, guanine in cytosine, and cytosine in guanine. The resulting “pre-RNA,” like the DNA from which it was generated, contains both exon and intron sequences. Next, the introns are physically excised from the pre-RNA molecule, in a process called “splicing,” to produce a messenger RNA (“mRNA”).

Following transcription, the resulting mRNA is “translated” into the encoded protein. Genes, and their corresponding mRNAs, encode proteins via three-nucleotide combinations called codons. Each codon corresponds to one of the twenty amino acids that make up all proteins or a “stop” signal that terminates protein translation. For example, the codon adenine-thymine-guanine (ATG, or UTG in the corresponding mRNA), encodes the amino acid methionine. The relationship between the sixty-four possible codon sequences and their corresponding amino acids is known as the genetic code.

Changes, or mutations, in the sequence of a gene can alter the structure as well as the function of the resulting protein. Small-scale changes include point mutations in which a change to a single nucleotide alters a single amino acid in the encoded protein. For example, a base change in the codon GCU to CGU changes an alanine in the encoded protein to an arginine. Larger scale variations include the deletion, rearrangement, or duplication of larger DNA segments, ranging from several hundreds to over a million nucleotides, and result in the elimination, misplacement, or duplication of an entire gene or genes. While some mutations have little or no effect on processes, others result in disease, or an increased risk of developing a particular disease. DNA sequencing is used in clinical diagnostic testing to determine whether a gene contains mutations associated with a particular disease or risk of a particular disease.

Nearly every cell contains an entire genome. DNA in the cell, called “native” or “genomic” DNA, is packaged into chromosomes. Chromosomes are complex structures of a single DNA molecule wrapped around proteins called histones.

Genomic DNA can be extracted from its cellular environment using a number of well-established laboratory techniques. A particular segment of DNA, such as a gene, can then be excised or amplified from the DNA to obtain the isolated DNA segment of interest. DNA molecules can also be synthesized in the laboratory. One type of synthetic DNA molecule is complementary DNA (“cDNA”). cDNA is synthesized from mRNA using complementary base pairing in a manner analogous to RNA transcription. The process results in a double-stranded DNA molecule with a sequence corresponding to the sequence of an mRNA produced by the body. Because it is synthesized from mRNA, cDNA contains only the exon sequences, and thus none of the intron sequences, from a native gene sequence.

An oligonucleotide is a short segment of RNA or DNA, typically comprising approximately thirty or fewer nucleotide bases. Oligonucleotides may be formed by the cleavage or division of longer RNA/DNA segments, or may by synthesized by polymerizing individual nucleotide precursors, such as by polymerase chain reaction (PCR) and/or other known techniques. Automated synthesis techniques such as PCR may allow the synthesis of oligonucleotides up to 160 to 200 nucleotide bases. With respect to PCR, an oligonucleotide is commonly referred to as a “primer,” which allows DNA polymerase to extend the oligonucleotide and replicate the complementary strand. The length of an oligonucleotide is typically denoted in terms of “mer.” By way of non-limiting example, an oligonucleotide having 25 nucleotide bases would be characterized as a 25-mer oligonucleotide. Because oligonucleotides readily bind to their respective complementary nucleotide, they may used as probes for detecting particular DNA or RNA. The oligonucleotides can be made with standard molecular biology techniques known in the art and disclosed in manuals such as Sambrook et al., Molecular Cloning: A Laboratory Manual, Cold Spring Harbor Laboratory Press, Cold Spring Harbor, N.Y., USA (1989) or conventional nucleotide phosphoramidite chemistry and commercially available synthesizer instruments. The oligonucleotides can be DNA or RNA. Also contemplated are the RNA equivalents of the oligonucleotides and their complements.

The term “primer” refers to an isolated single stranded oligonucleotide sequence capable of acting as a point of initiation for synthesis of a primer extension product which is complementary to the nucleic acid strand to be copied. The length and the sequence of the primer must be such that they allow to prime the synthesis of the extension products. A primer is about 5-50 nucleotides long, more specifically from 10 to 40 nucleotides long. Specific length and sequence will depend on the complexity of the required DNA or RNA targets, as well as on the conditions of primer use such as temperature and ionic strength.

The oligonucleotides used as primers or probes may also comprise nucleotide analogues such as phosphorothiates, alkylphosphorothiates or peptide nucleic acids or may contain intercalating agents.

As most other variations or modifications introduced into the original DNA sequences, these variations will necessitate adaptions with respect to the conditions under which the oligonucleotide should be used to obtain the required specificity and sensitivity. However the eventual results of hybridization will be essentially the same as those obtained with the unmodified oligonucleotides.

The introduction of these modifications may be advantageous in order to positively influence characteristics such as hybridization kinetics, reversibility of the hybrid-formation, biological stability of the oligonucleotide molecules, etc.

The two main pan-genus polymerase chain reaction (PCR) based detection methods may be used to survey the presence of rare and/or unknown variants of organisms. Both PCR assays have distinct advantages and disadvantages that differ from both a scientist and clinician perspective.

First, traditional PCR based detection methods utilize specific primers to amplify identifying sequences of an organism. Amplified products are visualized via gel electrophoresis and bands that are within a certain size range can be further analyzed by restriction enzyme digest or by sequence analysis. This approach has significant advantages due to the flexibility in choices of primer design. Primers can be designed intentionally to amplify entire groups of related organisms and the stringency can be controlled by altering the primer positions, primer degeneracy, and primer annealing temperatures. Having the flexibility to make few assumptions about the target organism could provide detection of rare or novel species, thus providing immediate benefit to clinicians and their patients.

However, this method is not without some weaknesses. Moderate stringency PCRs produces some potential positives that, upon sequence analysis, are identified as artifacts which give the false appearance of a positive PCR signal. Therefore, effort spent on sequencing these false bands was wasted. Furthermore, the fact that this method intentionally is of moderate stringency opposes finely tuned and optimized detection levels. Other PCR based methods could allow exceptionally sensitive detection down to even a single organismal genome in an entire patient sample. Thus far, it appears as if clinicians are willing to accept some loss of sensitivity for the increased chance at detecting rare or novel species.

Second, a newer PCR based detection technique utilizes quantitative PCR (qPCR) that uses either fluorescently labeled nucleotides or probes to spectroscopically measure the levels of amplified product. This technique requires highly optimized and stringent probes, thus reducing the probability of pan-genus detection. However, qPCR is sensitive enough to detect exceptionally low copy numbers of the organismal genome.

The term “probe” refers to isolated single stranded sequence-specific oligonucleotides which have a sequence which is complementary to the target sequence to be detected. Complementarity of the probe sequence to the target sequence is essential and complete for the central part of the probe (=core of the probe), where no mismatches to the target sequence are allowed. Towards the extremities (3′ or 5′) of the probe, minor variations in the probe sequence may sometimes occur, without affecting the species specific hybridization behavior of the probe. The “core sequence” of the probe is the central part, and represents more than 70%, more than 80%, most often more than 90% of the total probe sequence.

The probes disclosed herein specifically hybridize to Protomyxzoa Rhuematic for which they are designed. Throughout this document, the sequences of the probes are always represented from the 5′ end to the 3′ end. They are represented as single stranded DNA molecules. It should be understood however that these probes may also be used in their RNA form (wherein T is replaced by U), or in their complementary form.

Probes may be formed by cloning of recombinant plasmids containing inserts comprising the corresponding nucleotide sequences, if need be by cleaving the latter out from the cloned plasmids upon using the adequate nucleases and recovering them, e.g. by fractionation according to molecular weight. The probes according to the present invention can also be synthesized chemically, for instance by the conventional phospho-triester method.

Some of the probes disclosed herein have a length from about 10 to about 30 nucleotides. Variations are possible in the length of the probes and it should be clear that, since the central part of the probe is essential for its hybridization characteristics, possible deviations of the probe sequence versus the target sequence may be allowable towards head and tail of the probe, especially when longer probe sequences are used. These variant probes, should however always be evaluated experimentally, in order to check if they result in equivalent hybridization characteristics than the original probes.

The term “isolated” as used herein means that the oligonucleotides disclosed herein are isolated from the environment in which they naturally occur. In particular, it means that they are not an % more part of the genome of the respective species, and thus liberated from the remaining flanking nucleotides in the target region of the species. On the contrary, new (=heterologous) flanking regions may be added to the 3′ and/or 5′ end of the probe, in order to enhance its functionality. Functional characteristics possibly provided by said heterologous flanking sequences are e.g. ease of attachment to a solid support, ease of synthesis, ease of purification, labeling function etc.

The term “complementary” nucleic acid as used herein means that the nucleic acid sequences can form a perfect base-paired double helix with each other.

The term “specific hybridization” refers to a selective hybridization of the probes disclosed herein to the nucleic acids of Protomyxzoa Rhuematic to be detected (=target organism), and not to nucleic acids originating from strains belonging to other species (=non-target organisms). Specific hybridization in the context of the present disclosure also implies a selective hybridization of the disclosed probes to the target region of Protomyxzoa Rhuematic to be detected, and limits occasional “random” hybridization to other genomic sequences. Specificity is a feature which has to be experimentally determined. Although it may sometimes be theoretically predictable, specificity can only refer to those non-target organisms which have been tested experimentally.

The term “sample” represents any material possibly containing nucleic acids, which may have to be released from the cells. Preferably, the term “sample” refers to a clinical sample, such as a sample taken from blood, from the respiratory tract (sputum, bronchoalveolar lavage (BAL)), from cerebrospinal fluid (CSF), from the urogenital tract (vaginal secretions, urine), from the gastrointestinal tract (saliva, faeces) or biopsies taken from organs, tissue, ski, etc. The term “sample” may also refer to a sample of cultured cells, either cultured in liquid medium or on solid growth media. DNA present in said samples may be prepared or extracted according to any of the techniques known in the art.

The “target” material in these samples may be either genomic DNA or precursor ribosomal RNA of the organism to be detected (=target organism), or amplified versions thereof. These molecules are called target nucleic acids.

The term “isolated” oligonucleotide refers to an oligonucleotide that is found in a condition other than its native environment. In a preferred form, the oligonucleotide is substantially free from other nucleic acid sequences, such as other chromosomal and extrachromosomal DNA and RNA, that normally accompany or interact with it as found in its naturally occurring environment. The term “isolated” oligonucleotide also embraces recombinant oligonucleotides and chemically synthesized oligonucleotides.

The term “test sample” as used herein, means anything designated for testing for the presence of an organism and/or the nucleic acid of an organism. The test sample is, or can be derived from any biological source, such as for example, blood, blood plasma, cell cultures, tissues and mosquito samples. The test sample can be used directly as obtained from the source, or following a pre-treatment to modify the character of the sample. Thus, the test sample can be pre-treated prior to use by, for example, preparing plasma from blood, disrupting cells or viral particles, preparing liquids from solid materials, diluting viscous fluids, filtering liquids, distilling liquids, concentrating liquids, inactivating interfering components, adding reagents, and purifying nucleic acid.

Case Study—In Situ Hematologic Biofilm Communities in 3 Patients with ALS

Abstract—Spontaneous Amyotrophic Lateral Sclerosis, Lou Gehrig's disease (ALS) is a debilitating neurologic disease with an unknown cause and poor prognosis. Efforts to determine the etiology have not been conclusive. It is believed that ALS has an infectious trigger and the causative pathogen could be found in the peripheral circulation, and that the utilization of careful microscopic, histological, and molecular techniques could provide insight into the mechanism of disease. Suspecting that ALS is an infectious disease with great antibiotic resistance, the existence of a biofilm-based pathogen was postulated. Peripheral smears from three ALS patients were examined with a variety of stains and techniques. Molecular analysis of peripheral blood samples using broad fungal, prokaryotic and protozoan probes was done. Results indicated the presence of biofilm communities. Molecular analysis suggested the presence of protozoan, bacterial, and fungal organisms.

Case Report—Spontaneous Amyotrophic Lateral Sclerosis, Lou Gehrig's disease (ALS) is a debilitating neurologic disease with an unknown cause and poor prognosis. Recent work has demonstrated chronic cerebral venous insufficiency (CCVI) in Multiple Sclerosis (MS) patients (1). Clearance of CCVI membranous obstruction by percutaneous transluminal angioplasty results in clinical improvement in MS. It has been suggested that the same mechanism may be present in ALS (2). It is believed that ALS is an infectious disease with great antibiotic resistance, and the existence of a biofilm based pathogen was postulated. This biofilm could manifest as the macroscopic membranes visualized in the work by Zamboni.

Peripheral blood samples from three ALS patients were collected after obtaining informed consent. This study was approved by the Fry Laboratories institutional review committee. All three patients were diagnosed by a board certified neurologist. A number of techniques were utilized including Hoechst staining, modified May-Gr\ddot{υ}newald, Periodic Acid-Schiff Reagent, Giemsa and light microscopy techniques (3). Molecular analysis by PCR was done by using bacterial, fungal, and protozoan primers. Microscopic examination of stained smears revealed an abundance of epierythrocytic bacteria attached to peripheral red blood cells. These were 1-2 micrometers in diameter, coccoid and coccobacillary, and consistent with the description of Hemobartonella published in the human and veterinary literature (4).

Examination of histological preparations revealed amorphous clusters of material that on first impression appear to be artifact or ‘dirt’. Further examination with high power microscopy, Hoechst stain, Ethidium Bromide, or Giemsa staining revealed a mixed population of eukaryotic appearing organisms, adherent lymphocytes, and smaller bacterial shapes consistent with the organisms previously observed and described as Hemobartonella. The sizes of these clusters were 20-100 micrometers. PAS stain revealed a polysaccharide matrix found throughout contributing to cluster morphology. The composition and appearance of these clusters were consistent with previous published and observed biofilm communities (5).

DNA was extracted from peripheral blood and assessed by PCR using general and specific bacterial, fungal and protozoan primers. Analysis of PCR products by sequencing and BLAST confirmed mixed populations of organisms. These consisted of Proteobacteria (primarily Ralstonia spp.), fungi, human DNA, and evidence suggestive of protozoans. All three patients with ALS displayed similar findings.

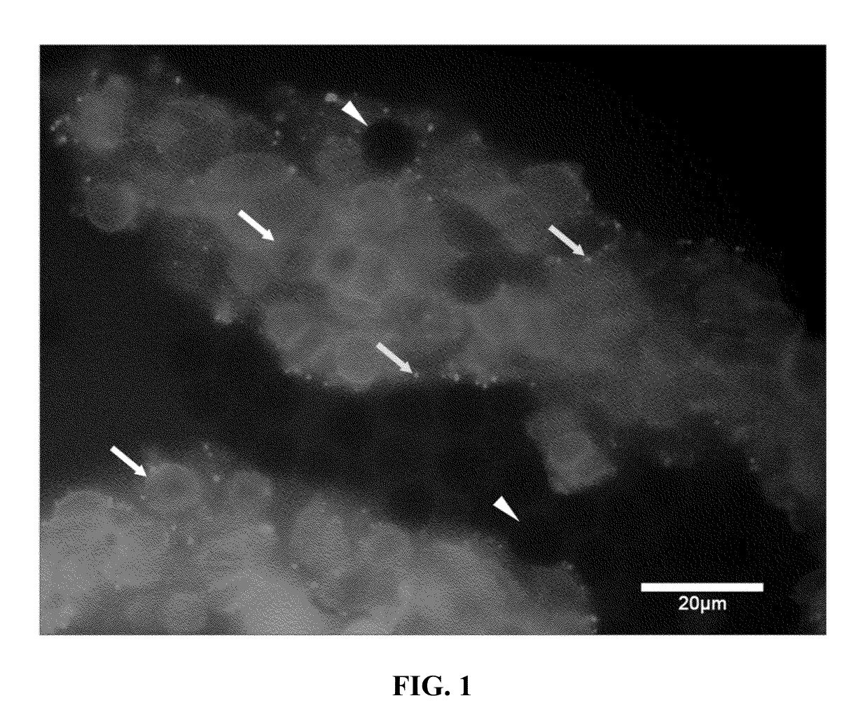

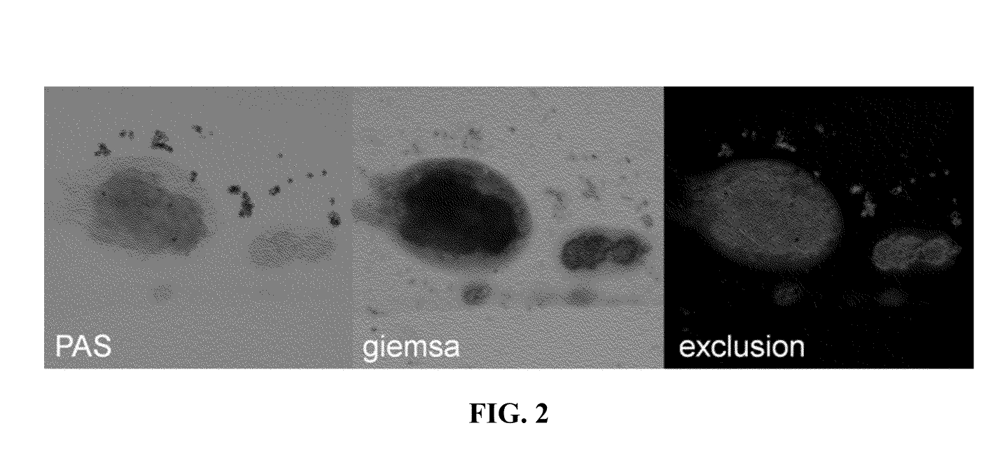

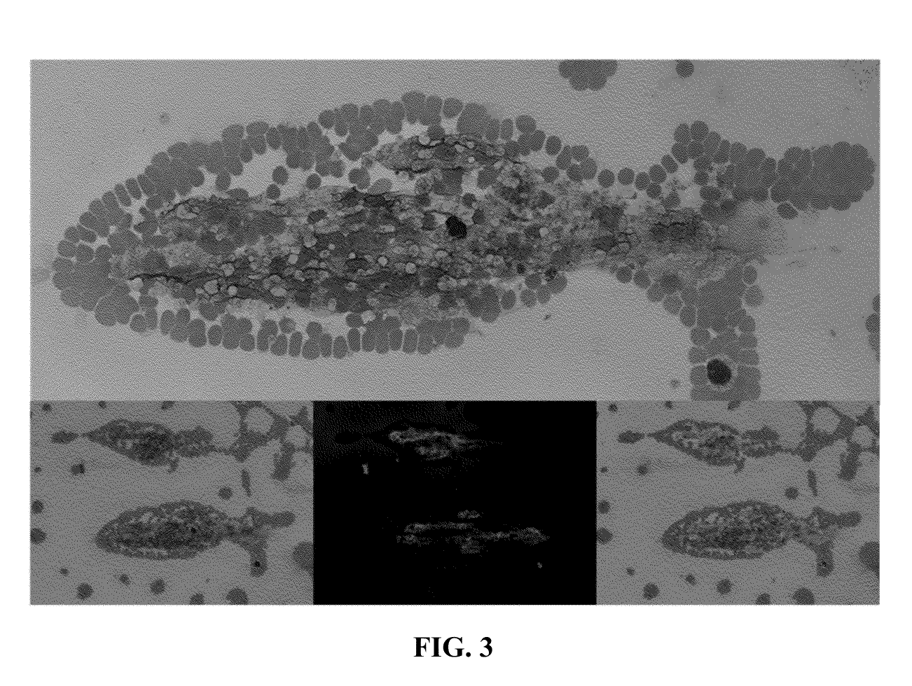

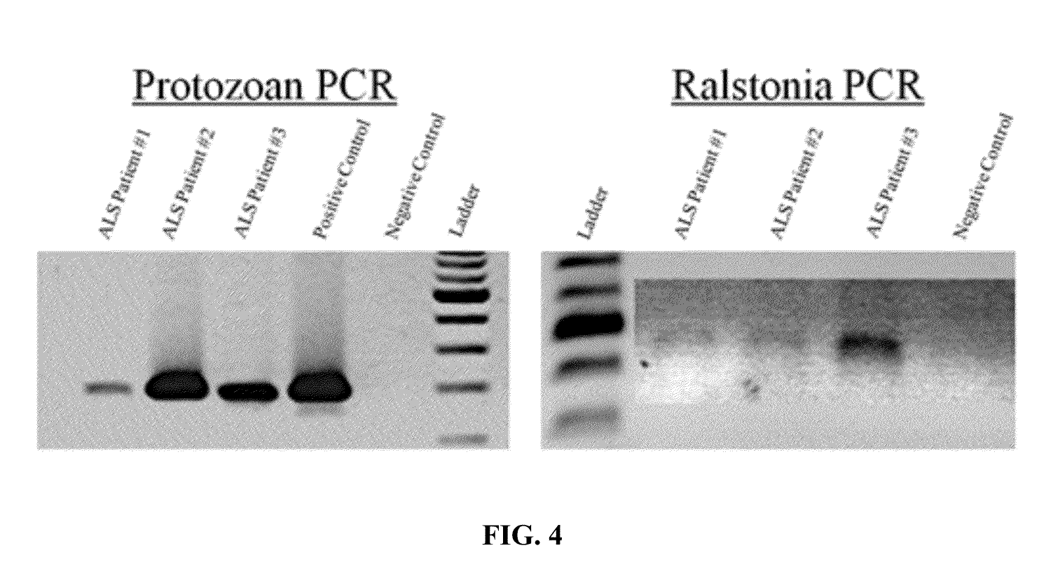

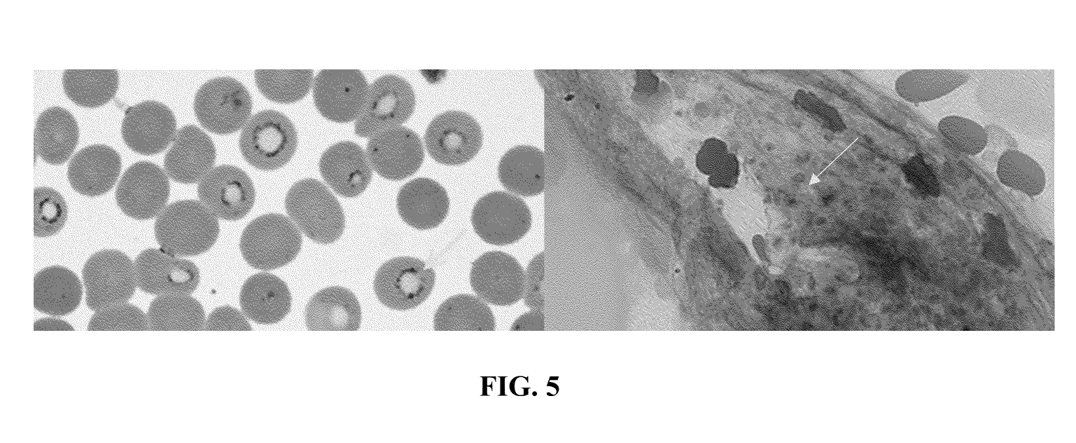

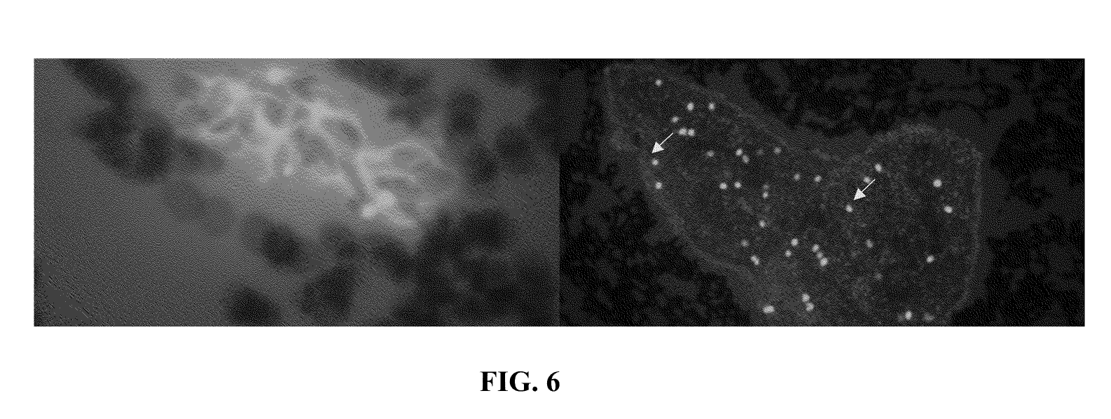

Microscopic study of stained blood smears from the 3 ALS patients revealed 1-2 μm diameter epierythrocytic bacteria, while fluorescent DNA staining techniques revealed large amorphous clusters, 20-100 μm in diameter, consisting of eukaryotic organisms, adherent lymphocytes, and bacteria (FIG. 1—Cluster fragment from ALS patient peripheral blood 1000×, excitation @ 620 nm; Ethidium Bromide and Hoechst stain; visible are multiple organisms from coccobaciili to larger eukaryotic organisms representative of a complex biofilm community). Additionally, Hoechst (FIG. 3) and PAS (FIG. 2) stains reveal that the extracellular matrix material associated with these clusters contain both DNA and polysaccharides. The morphology of these structures was consistent with what is observed in environmental biofilm communities. DNA extracted from peripheral blood was assessed by PCR using bacterial, fungal, and protozoan primers. Subsequent sequencing and analysis revealed a mixed population of organisms, including Proteobacteria (primarily Ralstonia spp.), fungi, and sequences suggestive of a novel protozoan species (Table 1 and FIG. 4). Microscopic observations combined with PCR results are consistent with a persistent biofilm community found in the peripheral blood of these patients with ALS.

Thus, observation under microscopy and PCR based methods are consistent with a protozoan foundation pathogen and primary biofilm former. It is believed that ALS may be caused by a foundation protozoan which produces a polysaccharide biofilm, hosting a communalistic environment for additional microorganisms. These clusters consist of a biofilm matrix and are freely circulating in the peripheral vascular system. Continued host lymphocytic response is evidenced by both PCR and microscopy. It is suspected that Fungi and Proteobacteria spp. are opportunistic members of this biofilm community. The postulated mechanism of disease is a gross obstructive sludging and macroscopic mechanical coagulation producing ischemia, retrograde venous flow, and poor nutrient supply to the surrounding tissues.

The concept of eukaryotic biofilms has been demonstrated recently in the case of the fungi P. carinii (6). Protozoan biofilms represent an emerging area in need of further study, particularly their role in ALS and other diseases. The identity of the protozoan foundation pathogen is Protomyxzoa Rhuematic described below.

References are as follows:
1. Bromberg M B. Pathogenesis of amyotrophic lateral sclerosis: a critical review. Curr Opin Neurol 1999; 12:581-588.
2. Ince P G, Lowe J, Shaw P J. Amyotrophic lateral sclerosis: current issues in classification, pathogenesis, and molecular pathology. Neuropathology and Applied Neurobiology 1998; 24:104-117.
3. Bancroft J D, Gamble M. Theory and practice of Histological Techniques, 6th ed. Philadelphia: Churchill Livingstone; 2007.
4. Willi B, Boretti F S, Tasker S, Meli M, Wengi N, et al. From Haemobartonella to hemoplasma: molecular methods provide new insights. Vet Microbiol 2007; 125:197-209.
5. Hall-Stoodley L, Stoodley P. Evolving concepts in biofilm infections. Cell Microbiol 2009; 11:1034-1043.
6. Cushion M T, Collins M S, Linke M J. Biofilm formation by Pneumocystis spp. Eukaryot Cell 2009; 8:197-206.

### Protomyxzoa Rhuematic: A Novel Infectious Organism; Missing Etiology in Chronic Disease

Rheumatic and inflammatory diseases have had a long history of links with infectious agents ranging from molecular mimicry effects to the direct activity of human pathogens. The following orphan diseases and conditions are of keen interest: Chronic Fatigue Syndrome; Fibromyalgia; Ulcerative Colitis; Gulf War Veterans Illness; Scleroderma; ALS (Lou Gehrig's Disease); Rheumatoid Arthritis; Parkinson's Disease; Osteoarthritis; Multiple Sclerosis; Crohn's Disease; and Autism.

Using a basic science approach and microscopic techniques Protomyxzoa Rhuematic (See FIG. 1) was found in patients with a wide range of chronic diseases. It is believed that Protomyxzoa Rhuematic is a novel hematologic biofilm-forming protozoan with Malaria-like and Babesia-like characteristics. Protomyxzoa Rhuematic is primarily hematogenous, lipid loving, complex (probably ‘Myxozoan’), and very drug resistant, but antiprotozoals and antihelminthics may be efficacious. Furthermore, research indicates that multiple species may be found cohabitating within the Protomyxzoa Rhuematic biofilm. Biofilm communities with Protomyxzoa Rhuematic as the foundation pathogen cause gross obstructive sludging resulting in macroscopic mechanical coagulation and retrograde venous flow. This results in ischemia and poor nutrient supply to surrounding tissues and chronic infection (chronic inflammatory response by lymphocytes).

Thus, initial results indicate that a variety of pathogenic bacteria and potential viruses are harbored within the biofilm matrix and may be pathogenic factors. Results are consistent with this novel organism having profound biofilm forming properties that are likely related to observed clinical significance in patients with chronic inflammatory diseases. It is not difficult to hypothesize that deficits in blood perfusion may contribute or exacerbate symptoms in these patients.

This research has transitioned from merely microscopic study to molecular characterization and genomic sequencing of Protomyxzoa Rhuematic. The existence of Protomyxzoa Rhuematic and continued study may be critically important to the treatment and outcome of patients with chronic inflammatory and neurologic diseases.

Identifying Sequences

The accompanying SEQUENCE LISTING (which is hereby incorporated herein by reference) identifies 8,454 sequences (SEQ ID NO:1 through SEQ ID NO:8454) that are representative for Protomyxzoa Rheumatic.

Advanced Detection

First, research microscopic techniques that were instrumental in the discovery of Protomyxzoa Rhuematic have been adapted for use as a clinical diagnostic assay. A proprietary Advanced Stain test is not only specifically designed to detect biofilm based infections in aqueous samples, but it has been rigorously shown to detect known pathogens from bacterial infections, to disseminated fungal infections, to protozoans such as Malaria and Babesia. The principle of this assay relies on the simple fact that infectious organisms have DNA. By adding specific DNA dye to the samples coupled with fluorescent microscopy, a direct visualization of hematologic infections can be documented for health care professional and physician use. Blood samples are particularly well suited for this type of staining technique because red blood cells do not have nuclear DNA, thus appearing as a dark backdrop against infectious organisms that shine brightly, with the patient white blood cells providing an excellent internal control for staining quality.

In addition to microscopy-based detection of biofilms by an Advanced Stain test, a Protomyxzoa Rhuematic PCR based test can be used. This assay provides information about the detectable levels of Protomyxzoa Rhuematic in a patient sample.

This suite of tools will assist health care professionals to accurately identify, monitor, and treat patients found to harbor Protomyxzoa Rhuematic.

### Compositions and Methods

There are many aspects of compositions and methods for detecting protozoan pathogen Protomyxzoa Rhuematic disclosed herein, of which one, a plurality, or all aspects may be used in any particular implementation.

It is to be understood that various implementations may be utilized, and compositional, as well as procedural, changes may be made without departing from the scope of this document. As a matter of convenience, various compositions and methods will be described using exemplary materials, sizes, specifications, and the like. However, this document is not limited to the stated examples and other configurations are possible and within the teachings of the present disclosure.

Implementations of the disclosed compositions and methods relate generally to oligonucleotides useful in methods for determining whether a sample contains Protomyxzoa Rhuematic. Although, any recombinant products such as peptides and the like are within the scope of this disclosure, which could also be used as diagnostics for markers or in immunological testing as antigens.

In one aspect, protozoan pathogen Protomyxzoa Rhuematic may be identified using any of the 5′-3′ sequences set forth in the SEQUENCE LISTING.

In another aspect, implementations of the disclosed compositions and methods relate generally to oligonucleotides, recombinant products such as peptides, and the like useful in methods for determining whether a sample contains protozoan pathogen Protomyxzoa Rhuematic, or has an increased likelihood of containing protozoan pathogen Protomyxzoa Rhuematic, a new genus and species of organism which is seen in conjunction with CFS, Fibromyalgia, the autoimmune diseases, ALS, MS, Parkinson's disease, Autism, and the like.

Isolated oligonucleotides (by way of non-limiting example, a forward primer, a reverse primer, or a probe such as a molecular beacon) are capable of detecting a unique biomarker for the protozoan pathogen Protomyxzoa Rhuematic species.

### Example 1

For the exemplary purposes of this disclosure, as one example, the following 5′-3′ sequence (SEQUENCE NODE 0), from the protozoan pathogen Protomyxzoa Rhuematic species, can be detected by real time quantitative PCR using the following primers and probe (PRIMERS/PROBES) to amplify and detect a 153 base pair fragment with the following PCR conditions (CONDITIONS).

(Sequence Node 0)

<NODE_{—}0_length_{—}429_cov_{—}3.000000;DNA;>

CCATGCATGTCTAAGTATAAGCACTTATACAGTGAAACTGCGAATGGCTCA
TTATATCAGTTATAGTTTATT
TGATAGTCCCTACTACTTGGATAACCGTAGTAATTCTAGAGCTAATACATG
CGCTAACTCCTGACCTCACGG
AAAGGATGTATTTATTAGATACAACCAACCTTGTTTGGTGAATCATAATAA
CTGAGCGAACCGCATGCTTCG
GCGGCGGTGGTTCATTCAAGTTTCTGACCTATCAGCTTTCGATGGTAGGGT
ATTGGCCTACCATGGCGTTAA
CGGGTAACGGAGAATTAGGGTTCGATTCCGGAGAGGGAGCCTGAGAGATGG
CTACCACATCCAAGGAAGGCA
GCAGGCGCGTAAATTACCCAATCCTGACACAGGGAGGTAGTGACAAGAAAT
AACAATGCGGAGCCTTCG

(Primers/Probes)

FL1953_F2
(5′-ATGGCTCATTATATCAGTTATAGT-3′)
FL1953_R1
(5′-GTTATTATGATTCACCAAACAAG-3′)
FL1953_PROBE
(5′-FAM-ACATCCTTT/ZEN/CCGTGAGGTCAGGAGTT-
3IABkFQ-3′)

(Conditions)

37° C. 15 min

95° C. 10 min

50 cycles of: 95° C. 30 sec; 60° C. 1 min; 72° C. 30 sec

### Example 2

For the exemplary purposes of this disclosure, as another example, standard PCR using the following (PRIMERS-STANDARD PCR) can amplify a 196 base pair fragment, also for the sequence above (SEQUENCE NODE 0), that can be visualized by gel electrophoresis.

(Primers-Standard PCR)

FL1953_F1
(5′-CCATGCATGTCTAAGTATA-3′)
FL1953_R1
(5′-GTTATTATGATTCACCAAACAAG-3′)

In still another aspect, test kits useful for detecting Protomyxzoa Rhuematic from a sample may comprise at least one oligonucleotide disclosed in this document. The test kits may contain one or more pairs of oligonucleotides such as the primer pairs disclosed herein, or one or more oligonucleotide sets as disclosed herein. The assay kit can further comprise the four-deoxynucleotide phosphates (dATP, dGTP, dCTP, dTTP) and an effective amount of a nucleic acid polymerizing enzyme. A number of enzymes are known in the art which are useful as polymerizing agents. These include, but are not limited to E. coli DNA polymerase I, Klenow fragment, bacteriophage T7 RNA polymerase, reverse transcriptase, and polymerases derived from thermophilic bacteria, such as Thermus aquaticus. The latter polymerases are known for their high temperature stability, and include, for example, the Taq DNA polymerase I. Other enzymes such as Ribonuclease H can be included in the assay kit for regenerating the template DNA. Other optional additional components of the kit include, for example, means used to label the probe and/or primer (such as a fluorophore, quencher, chromogen, etc.), and the appropriate buffers for reverse transcription, PCR, or hybridization reactions. The kit may also contain instructions for carrying out the methods.

In yet another aspect, methods useful for detecting protozoan pathogen Protomyxzoa Rhuematic from one or more samples may comprise aligning nucleotide sequences pair wise and determining the percent identities (percentage of identical matches) between universal or specific primers and the sample to be tested. In particular implementations, a reaction mixture or a kit may be provided comprising an isolated oligonucleotide (a forward primer, in particular implementations). In other particular implementations, a second isolated oligonucleotide, different than the first isolated oligonucleotide (a reverse primer, in particular implementations) may be provided. The primers are capable of hybridizing under highly stringent hybridization conditions to a polynucleotide consisting of any one of the nucleotide sequences set forth in SEQ ID NO:1 through SEQ ID NO:8454.

Methods useful for detecting protozoan pathogen Protomyxzoa Rhuematic from one or more samples may further comprise a method for determining whether a sample (by way of non-limiting examples, a blood sample, or other a biological sample, such as a swab specimen) contains protozoan pathogen Protomyxzoa Rhuematic or has an increased likelihood of containing protozoan pathogen Protomyxzoa Rhuematic, wherein the method comprises the following: (a) providing a vessel containing a composition, wherein the composition contains a primer and a nucleic acid from the sample, wherein the composition is capable of amplifying by a polymerase chain reaction a segment of the nucleic acid to produce an amplicon, wherein production of the amplicon is primed by the primer, wherein the primer is capable of hybridizing under highly stringent hybridization conditions to a polynucleotide consisting of any one of the nucleotide sequences set forth in SEQ ID NO:1 through SEQ ID NO:8454; (b) incubating the vessel under conditions allowing production of the amplicon if the sample contains protozoan pathogen Protomyxzoa Rhuematic, and (c) determining that the sample contains protozoan pathogen Protomyxzoa Rhuematic if the amplicon is detected, or that the sample has an increased likelihood of containing protozoan pathogen Protomyxzoa Rhuematic if the amplicon is detected, or otherwise determining that the sample does not contain protozoan pathogen Protomyxzoa Rhuematic if the amplicon is not detected or that the sample does not have an increased likelihood of containing protozoan pathogen Protomyxzoa Rhuematic if the amplicon is not detected.

Alternatively, in step (b) of the method above, the vessel may contain an oligonucleotide probe (by way of non-limiting example, a molecular beacon) capable of detecting the amplicon if the amplicon is produced in (b).

The amplifying step can be performed using any type of nucleic acid template-based method, such as PCR technology.

PCR technology relies on thermal strand separation followed by thermal dissociation. During this process, at least one primer per strand, cycling equipment, high reaction temperatures and specific thermostable enzymes are used (U.S. Pat. Nos. 4,683,195 and 4,883,202). Alternatively, it is possible to amplify the DNA at a constant temperature (Nucleic Acids Sequence Based Amplification (NASBA) Kievits, T., et al., J. Virol Methods, 1991; 35, 273-286; and Malek, L. T., U.S. Pat. No. 5,130,238; T7 RNA polymerase-mediated amplification (TMA) (Giachetti C, et al. J Clin Microbiol 2002 July; 40(7):2408-19; or Strand Displacement Amplification (SDA), Walker, G. T. and Schram, J. L., European Patent Application Publication No. 0 500 224 A2; Walker, G. T., et al., Nuc. Acids Res., 1992; 20, 1691-1696).

Amplified nucleic acid can be detected using a variety of detection technologies well known in the art. For example, amplification products may be detected using agarose gel by performing electrophoresis with visualization by ethidium bromide staining and exposure to ultraviolet (UV) light, by sequence analysis of the amplification product for confirmation, or hybridization with an oligonucleotide probe.

The oligonucleotide probe may be labeled with a detectable label. The detectable label can be any molecule or moiety having a property or characteristic that is capable of detection, such as, for example, radioisotopes, fluorophores, chemiluminophores, enzymes, colloidal particles, and fluorescent microparticles.

Probe sequences can be employed using a variety of methodologies to detect amplification products. Generally all such methods employ a step where the probe hybridizes to a strand of an amplification product to form an amplification product/probe hybrid. The hybrid can then be detected using labels on the primer, probe or both the primer and probe. Examples of homogeneous detection platforms for detecting amplification products include the use of FRET (fluorescence resonance energy transfer) labels attached to probes that emit a signal in the presence of the target sequence. “TaqMan” assays described in U.S. Pat. Nos. 5,210,015; 5,804,375; 5,487,792 and 6,214,979 (each of which is herein incorporated by reference) and Molecular Beacon assays described in U.S. Pat. No. 5,925,517 (herein incorporated by reference) are examples of techniques that can be employed to detect nucleic acid sequences. With the “TaqMan” assay format, products of the amplification reaction can be detected as they are formed or in a so-called “real time” manner. As a result, amplification product/probe hybrids are formed and detected while the reaction mixture is under amplification conditions.

For example, the PCR probes may be TaqMan® probes that are labeled at the 5′ end with a fluorophore and at the 3′-end with a quencher molecule. Suitable fluorophores and quenchers for use with TaqMan® probes are disclosed in U.S. Pat. Nos. 5,210,015, 5,804,375, 5,487,792 and 6,214,979 and WO 01/86001 (Biosearch Technologies). Quenchers may be Black Hole Quenchers disclosed in WO 01/86001.

Nucleic acid hybridization can be done using techniques and conditions known in the art. Specific hybridization conditions will depend on the type of assay in which hybridization is used. Hybridization techniques and conditions can be found, for example, in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York) and Sambrook et al. (1989) Molecular Cloning. A Laboratory Manual (2d ed., Cold Spring Harbor Laboratory Press, Plainview, N.Y.).

Hybridization of nucleic acid may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified. Alternatively, stringency conditions can be adjusted to allow some mismatching in sequences so that lower degrees of similarity are detected.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30.degree. C. for short probes (e.g., 10 to 50 nucleotides) and at least about 60.degree. C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37.degree. C., and a wash in 1.times. to 2.times.SSC (20.times.SSC=3.0 M NaCl/0.3 M trisodium citrate) at 50 to 55.degree. C. Exemplary moderate stringency conditions include hybridization in 40 to 45% formamide, 1.0 M NaCl, 1% SDS at 37.degree. C., and a wash in 0.5.times. to 1.times.SSC at 55 to 60.degree. C. Exemplary high stringency conditions include hybridization in 50% formamide, 1 M NaCl, 1% SDS at 37.degree. C., and a wash in 0.1.times.SSC at 60 to 65.degree. C. The duration of hybridization is generally less than about 24 hours, usually about 4 to about 12 hours, or less depending on the assay format.

It should be noted that the oligonucleotides of this disclosure can be used as primers or probes, depending on the intended use or assay format. For example, an oligonucleotide used as a primer in one assay can be used as a probe in another assay. The grouping of the oligonucleotides into primer pairs and primer/probe sets reflects certain implementations only. However, the use of other primer pairs comprised of forward and reverse primers selected from different preferred primer pairs is specifically contemplated.

Isolation of the DNA sequences described above and in this disclosure from packed red blood samples of patients diagnosed with a disease for example may be accomplished according to the methods disclosed herein.

By way of non-limiting example, the amplified bacterial PCR product may be purified using commercially-available purification kits such as, by way of non-limiting example, the QIAquick® PCR purification kit manufactured by Quiagen, Inc. Sequencing may be performed with the primers described previously (Gerard 1997) using commercially-available capillary sequencers such as, by way of non-limiting example, a 3730 capillary sequencer manufactured by Applied Biosystems. In addition, the sequences may be analyzed using commercially-available sequence databases such as, by way of non-limiting example, the GenBank sequence database (BLASTN 2.2.8 program).

By way of other examples, the detection of Protomyxzoa Rhuematic by PCR can be achieved by the following methods: A. An expanded extraction followed by a Protomyxzoa Rhuematic specific PCR (C.); B. A sample enrichment, subsequent expanded extraction followed by a Protomyxzoa Rhuematic specific PCR (C.); and C. Protomyxzoa Rhuematic specific PCR.

A. Expanded Extraction Method

The following expanded extraction procedure is an “extreme” extraction procedure. Most other organisms and DNA would be destroyed.

This method also requires the Protomyxzoa Rhuematic specific PCR discussed below in section C. for reproducible detection of the Protomyxzoa Rhuematic genomic fragment.

The steps are as follows:

1. 750 μL of Qiagen Buffer AL is measured using a P1000 and dispensed into a labeled Zymo Research ZR Bashing Bead Lysis Tube.

2. Next, 200 μL of whole EDTA preserved blood is measured using a P200 and added to the same tube.

3. The screw cap of the Bashing Bead Lysis Tube is secured tightly and the sample is briefly vortexed.

4. The sample is placed in a centrifuge and briefly spun at 8000 rpm (˜6000 g) to remove solution from the screw cap.

5. 40 μL of reconstituted Proteinase K (>600 mAU/mL) is added to the sample in the Bashing Bead Lysis Tube. Note: The brand of Proteinase K does not appear to make any difference on efficiency of extraction and Qiagen's proprietary Protease may be substituted in most circumstances.

6. The screw cap of the Bashing Bead Lysis Tube is secured tightly and the sample is briefly vortexed.

7. The sample is then incubated for 5 minutes at 56° C. on a heating block.

8. The sample is then vortexed at maximum speed for 30 second.

9. The sample is then incubated for 5 minutes at 56° C. on a heating block.

10. The sample is then vortexed at maximum speed for 5 second.

11. The sample is allowed to rest for 5 second at room temperature.

12. Steps 10 and 11 are repeated 5 times. Proceed to step 13 after the final repetition of step 11.

13. The sample is then incubated for 2 minutes at 56° C. on a heating block.

14. The sample is then vortexed at maximum speed for 5 minutes using a tube holder.

15. The sample is then incubated for 5 minutes at 56° C. on a heating block.

16. The sample is then vortexed at maximum speed for 1 minute.

17. The sample is then incubated for 5 minutes at 56° C. on a heating block.

18. The sample is then vortexed at maximum speed for 1 minute. Note: Additional vortexing and incubation steps may improve sample recovery, but the preceding steps represent a minimal series of steps.

19. The sample is placed in a centrifuge and briefly spun at 8000 rpm (˜6000 g) to remove solution from the screw cap.

20. Prepare a Zymo Research Zymo-Spin IV Spin Filter by snapping off and removing the flow through plug, place the Spin Filter into the provided collection tube, and label the accompanying orange screw cap.

21. 600 μL of the sample is decanted using a P1000 and dispensed into the Spin Filter and the accompanying orange screw cap should be affixed securely. Note: Avoid aspirating beads from the Bashing Bead Lysis tube by moving the tip in a circular motion while decanting the sample as the beads may block the pipette tip from functioning.

22. The Spin Filter is placed in a centrifuge and spun at 8000 rpm (˜6000 g) for 1 minute.

23. 3004 of molecular biology grade 100% ethanol is added to the resulting flow through in the collection tube and mixed by repeated pipetting.

24. 7004 of the flow though and ethanol mix is added to a labeled QIAamp Mini Spin Column (in a 2 mL collection tube) using a P1000 without wetting the rim.

25. The cap is gently closed and the tube is placed in a centrifuge and spun at 8000 rpm (˜6000 g) for 1 minute.

26. The Spin Column is placed in a new 2 mL collection tube and the old collection tube with resulting filtrate is discarded.

27. The Spin Column is gently opened and 5004 of Buffer AW1 is added using a P1000 without wetting the rim.

28. The cap is gently closed and the tube is place in a centrifuge and spun at 8000 rpm (˜6000 g) for 1 minute.

29. The Spin Column is placed in a new 2 mL collection tube and the old collection tube with resulting filtrate is discarded.

30. The Spin Column is gently opened and 500 μL of Buffer AW2 is added using a P1000 without wetting the rim.

31. The cap is gently closed and the tube is placed in a centrifuge and spun at maximum rpm (˜20,000 g) for 1 minute.

32. The Spin Column is placed in a new 1.5 mL microcentrifuge tube and the old collection tube with resulting filtrate is discarded.

33. 30 μL of Buffer AE is added to the Spin Column and incubated for 2 minutes at room temperature. Note: Alteration of elution volume may increase template concentration.

34. After incubation the sample is placed in a centrifuge and spun at 8000 rpm (˜6000 g) for 1 minute.

35. The Spin Column is discarded. The resulting labeled and dated tube contains purified DNA from the starting blood sample.

36. The sample concentration may be determined by Nano-Drop or traditional spectrophotometer methods. Note: Expected concentration ranges between 5 ng/μL to 50 ng/μL, depending on the state and quality of the blood sample.

37. For short term storage the DNA sample may be kept at −20° C. For long term storage the DNA sample should be kept at −70° C.

38. Proceed to section C. below for PCR conditions and methods.

B. Combined Sample Enrichment and Expanded Extraction Method

This method requires both a sample enrichment method and expanded extraction method as discussed above in section A. in addition to the Protomyxzoa Rhuematic specific PCR discussed below in section C. for reproducible detection of the Protomyxzoa Rhuematic genomic fragment.

The steps are as follows:

1. Measure out 1.5 mL of well mixed human blood (EDTA preserved blood, although other blood preservation methods and possibly serum may work) into a new labeled 1.5 mL Eppendorf tube.

2. Place the tube into the microcentrifuge and spin at 300 g for 15 minutes.

3. Carefully remove tube and decant the supernatant off the sample into a new labeled 1.5 mL Eppendorf tube using a P200. Be careful not to aspirate any of the blood cells. Leaving some of the supernatant reduces the introduction of any blood cells.

4. Place the tube into the microcentrifuge and spin at 14,000 or maximum g for 10 minutes.

5. Carefully remove the tube and decant the supernatant off the pellet. Be careful to not disturb the pellet that formed in the bottom of the tube. Discard the supernatant.

6. Add 200 μL of molecular biology grade water to the pellet and pulse vortex until the pellet is fully resuspended.

7. 750 μL of Qiagen Buffer AL is measured using a P1000 and dispensed into a labeled Zymo Research ZR Bashing Bead Lysis Tube.

8. All 2004 of the resuspended sample is added to the same tube.

9. Follow steps #3 to #38 in Section A. discussed above.

C. Protomyxzoa Rhuematic PCR Detection Method

This method is used for reproducible detection of the Protomyxzoa Rhuematic genomic fragment. The steps are as follows:

1. A master mix is formulated using the following reagents added in the following order in the listed volumes per 10 μL reaction using standard PCR techniques.

i. 3.484 of H2O

ii. 54 of Sigma Extract-N-Amp Enzyme Mix

iii. 0.264 of FL1953_F primer (5′-CCATGCATGTCTAAGTATA-3′)

iv. 0.264 of FL1953_R primer (5′-GTTATTATGATTCACCAAACAAG-3′)

2. Once the master mix is thoroughly mixed and dispensed into individual PCR tubes 1 μL of the extracted DNA sample from either Section A. or Section B. above is added to each PCR tube with one negative control per 10 samples and minimally one positive control. Note: It is highly recommended that a separate set of PCRs accompany these samples using a pair of universal primers, such as 18S, to ensure that PCR inhibitors are not present in the sample.

3. The resulting 10 μL volume is thoroughly mixed and placed in a validated PCR thermocycler using the following PCR reaction conditions:

i. 95° C. for 2 minutes

ii. 95° C. for 30 seconds

iii. 55° C. for 30 seconds

iv. 72° C. for 20 seconds

v. Repeat steps ii, iii, and iv 50 times

vi. 72° C. for 7 minutes

vii. Hold at 4° C., until sample is to be analyzed

4. Resulting PCR products are visualized by gel electrophoresis on a 2% gel and stained with ethidium bromide. A positive result corresponds to a band that migrates at approximately 190 bp identically with the positive control band. Negative samples should be re-extracted by methods in Section A. or Section B. above and tested again by PCR to confirm the negative result.

The aspects/implementations outlined here, and many others, will become readily apparent to those of ordinary skill in the art from this disclosure. Those of ordinary skill in the art will readily understand the versatility with which this disclosure may be applied.

In places where the description above refers to particular implementations of compositions and methods for detecting protozoan pathogen Protomyxzoa Rhuematic, it should be readily apparent that a number of modifications may be made without departing from the spirit thereof and that these implementations may be alternatively applied. The accompanying CLAIMS are intended to cover such modifications as would fall within the true spirit and scope of the disclosure set forth in this document. The presently disclosed implementations are, therefore, to be considered in all respects as illustrative and not restrictive, the scope of the disclosure being indicated by the appended CLAIMS rather than the foregoing DESCRIPTION. All changes that come within the meaning of and range of equivalency of the CLAIMS are intended to be embraced therein.

For example, any recombinant products such as peptides and the like are within the scope of this disclosure, which could also be used as diagnostics for markers or in immunological testing as antigens.

1-2. (canceled)

3. A method for determining whether a sample contains or has an increased likelihood of containing Protomyxzoa Rheumatica comprising:
providing a vessel containing a composition, wherein the composition contains at least one of a first primer and a second primer, and a nucleic acid from the sample, wherein the composition is capable of amplifying, by a polymerase chain reaction, a segment of the nucleic acid to produce an amplicon, wherein production of the amplicon is primed by at least one of the first and second primers,
wherein the first primer is capable of hybridizing under highly stringent hybridization conditions to a polynucleotide consisting of the nucleotide sequence of one of:
any one of the nucleotide sequences set forth in SEQ ID NO:1 through SEQ ID NO:8454;
ATGGCTCATTATATCAGTTATAGT SEQ ID NO:8455; and
CCATGCATGTCTAAGTATA SEQ ID NO:8456; and
wherein the second primer is capable of hybridizing under highly stringent hybridization conditions to a polynucleotide consisting of the nucleotide sequence of one of:
any one of the nucleotide sequences set forth in SEQ ID NO:1 through SEQ ID NO:8454; and
GTTATTATGATTCACCAAACAAG SEQ ID NO:8457;
incubating the vessel under conditions allowing production of the amplicon if the sample contains Protomyxzoa Rheumatica; and
determining that the sample contains pathogen Protomyxzoa Rheumatica if the amplicon is detected or that the sample has an increased likelihood of containing pathogen Protomyxzoa Rheumatica if the amplicon is detected, or determining that the sample does not contain Protomyxzoa Rheumatica if the amplicon is not detected or that the sample does not have an increased likelihood of containing Protomyxzoa Rheumatica if the amplicon is not detected.

4. The method of claim 3, wherein the first primer is capable of hybridizing under highly stringent hybridization conditions to a polynucleotide consisting of the nucleotide sequence of ATGGCTCATTATATCAGTTATAGT SEQ ID NO:8455, wherein the second primer is capable of hybridizing under highly stringent hybridization conditions to a polynucleotide consisting of the nucleotide sequence of GTTATTATGATTCACCAAACAAG SEQ ID NO:8457, and wherein the vessel contains an oligonucleotide probe capable of detecting the amplicon if the amplicon is produced, the probe consisting of the nucleotide sequence of FAM-ACATCCTTT/ZEN/CCGTGAGGTCAGGAGTT-3IABkFQ SEQ ID NO:8458.

5. The method of claim 3, wherein the sample is an extracted sample obtained by one of an expanded extraction method and an enrichment, expanded extraction method.

6. The method of claim 3, wherein the polymerase chain reaction comprises hot start polymerase chain reaction.

7. The method of claim 3, wherein the step of determining comprises aligning nucleotide sequence pairs.

8. The method of claim 3, wherein the step of determining comprises determining a percent identities between one or more primers and the sample.

9. The method of claim 3, wherein the polymerase chain reaction comprises qPCR.

10. The method of claim 9, wherein the qPCR utilizes fluorescently labeled nucleotides to measure levels of amplified product.

11. The method of claim 3, wherein the step of determining comprises use of a probe.

12. The method of claim 11, wherein the probe comprises DNA molecules.

13. The method of claim 11, wherein the probe comprises RNA molecules.

14. The method of claim 3, wherein the sample is extracted using general and specific bacterial, fungal, and protozoal primers.

15. The method of claim 3, further comprising a step of determining mixed populations of organisms.

16. The method of claim 15, wherein the step of determining mixed populations of organisms comprises using bacterial, fungal, and protozoal primers.

17. The method of claim 3, further comprising a step of determining whether bacteria is present in the sample.

18. The method of claim 3, wherein the sample comprises a biofilm matrix.

19. The method of claim 3, wherein the sample is collected from a patient suspected of having a chronic inflammatory or neurological disorder.

20. The method of claim 3, wherein the step of detecting comprises one or more of agarose gel, sequence analysis of the amplification product for confirmation, and hybridization with an oligonucleotide probe.

21. The method of claim 3, wherein the step of detecting comprises detecting a label.

22. The method of claim 3, wherein the step of detecting comprises fluorescence resonance energy transfer.

23. The method of claim 3, further comprising a step of hybridizing under stringent conditions.

24. The method of claim 23, wherein the stringent conditions comprise use of a destabilizing agent.

25. The method of claim 24, wherein the destabilizing agent comprises formamide.

26. The method of claim 3, wherein the method comprises an expanded extraction followed by a Protomyxzoa Rheumatica specific PCR; sample enrichment, subsequent expanded extraction followed by a Protomyxzoa Rheumatica specific PCR; or Protomyxzoa Rheumatica specific PCR.

27. The method of claim 3, wherein the method comprises use of the first primer and the second primer.
